SEQUENCE LISTING (47 sequences)

SEQ ID NO: 1 - protein, 20 aa, Homo sapiens
DVNYAFLHATDLLPACDGER

SEQ ID NO: 2 - protein, 20 aa, Homo sapiens
SNMYAMMIARFKMFPEVKEK

SEQ ID NO: 3 - protein, 20 aa, Homo sapiens
NWELADQPQNLEEILMHCQT

SEQ ID NO: 4 - protein, 20 aa, Homo sapiens
TLKYAIKTGHPRYFNQLSTG

SEQ ID NO: 5 - protein, 20 aa, Homo sapiens
PRYFNQLSTGLDMVGLAADW

SEQ ID NO: 6 - protein, 20 aa, Homo sapiens
TYEIAPVFVLLEYVTLKKMR

SEQ ID NO: 7 - protein, 20 aa, Homo sapiens
FFRMVISNPAATHQDIDFLI

SEQ ID NO: 8 - protein, 14 aa, Homo sapiens
ILIKCDERGKMIPS

SEQ ID NO: 9 - protein, 14 aa, Homo sapiens
LGIGTDSVILIKCD

SEQ ID NO: 10 - protein, 14 aa, Homo sapiens
LAFLQDVMNILLQY

SEQ ID NO: 11 - protein, 14 aa, Homo sapiens
YDLSYDTGDKALQC

SEQ ID NO: 12 - protein, 14 aa, Homo sapiens
VSYQPLGDKVNFFR

SEQ ID NO: 13 - protein, 14 aa, Homo sapiens
LAADWLTSTANTNM

SEQ ID NO: 14 - protein, 14 aa, Homo sapiens
LLYGDAEKPAESGG

SEQ ID NO: 15 - protein, 14 aa, Homo sapiens
VNYAFLHATDLLPA

SEQ ID NO: 16 - protein, 14 aa, Homo sapiens
LLQYVVKSFDRSTK

SEQ ID NO: 17 - protein, 14 aa, Homo sapiens
FTYEIAPVFVLLEY

SEQ ID NO: 18 - protein, 14 aa, Homo sapiens
LEYVTLKKMREIIG

SEQ ID NO: 19 - protein, 14 aa, Homo sapiens
NMYAMMIARFKMFP

SEQ ID NO: 20 - protein, 14 aa, Homo sapiens
KIWMHVDAAWGGGL

SEQ ID NO: 21 - protein, 14 aa, Homo sapiens
WGGGLLMSRKHKWK

SEQ ID NO: 22 - protein, 14 aa, Homo sapiens
EGYEMVFDGKPQHT

SEQ ID NO: 23 - protein, 14 aa, Homo sapiens
RYFNQLSTGLDMVG

SEQ ID NO: 24 - protein, 14 aa, Homo sapiens
WLTSTANTNMFTYE

SEQ ID NO: 25 - protein, 14 aa, Homo sapiens
TANTNMFTYEIAPV

SEQ ID NO: 26 - protein, 14 aa, Homo sapiens
LVSATAGTTVYGAF

SEQ ID NO: 27 - protein, 14 aa, Homo sapiens
YIPPSLRTLEDNEE

SEQ ID NO: 28 - protein, 14 aa, Homo sapiens
VISNPAATHQDIDF

SEQ ID NO: 29 - protein, 25 aa, Homo sapiens
GMAALPRLIAFTSEHSHFSLKKGAA

SEQ ID NO: 30 - protein, 20 aa, Homo sapiens
ERGKMIPSDLERRILEAKQK

SEQ ID NO: 31 - protein, 8 aa, Homo sapiens
XPEVKTKZ

SEQ ID NO: 32 - protein, 8 aa, Homo sapiens
XPEVKEKZ

SEQ ID NO: 33 - protein, 14 aa, Homo sapiens
SNPAATHQDIDFLI

SEQ ID NO: 34 - DNA, 27 bp, Homo sapiens
  CDS (1)..(27)
ggcggaagccaaggaaatctcatcttt

SEQ ID NO: 35 - protein, 9 aa, Homo sapiens
GGSQGNLIF

SEQ ID NO: 36 - DNA, 24 bp, Homo sapiens
  CDS (1)..(24)
aacagagatgacaagatcatcttt

SEQ ID NO: 37 - protein, 8 aa, Homo sapiens
NRDDKIIF

SEQ ID NO: 38 - DNA, 21 bp, Homo sapiens
  CDS (1)..(21)
agcaatcagccccagcatttt

SEQ ID NO: 39 - protein, 7 aa, Homo sapiens
SNQPQHF

SEQ ID NO: 40 - DNA, 21 bp, Homo sapiens
  CDS (1)..(21)
agctacaatgagcagttcttc

SEQ ID NO: 41 - protein, 7 aa, Homo sapiens
SYNEQFF

SEQ ID NO: 42 - DNA, 12 bp, Homo sapiens
  CDS (1)..(12)
agtgcgggttgg

SEQ ID NO: 43 - protein, 4 aa, Homo sapiens
SAGW

SEQ ID NO: 44 - DNA, 18 bp, Homo sapiens
  CDS (1)..(18)
agcttggatgcgagcggg

SEQ ID NO: 45 - protein, 6 aa, Homo sapiens
SLDASG

SEQ ID NO: 46 - DNA, 20 bp, Artificial Sequence
  Description of Artificial Sequence synthetic primer
cactgaagatccatcatctg

SEQ ID NO: 47 - DNA, 20 bp, Artificial Sequence
  Description of Artificial Sequence synthetic primer
tagaggatggtggcagacag

(19) United States
(12) Patent Application Publication
(10) Pub. No.: US 2002/0114816 A1
(43) Pub. Date: Aug. 22, 2002

(54) AUTOREACTIVE PEPTIDES FROM HUMAN GLUTAMIC ACID-DECARBOXYLASE (GAD)

(76) Inventors: JOSEF ENDL, WEILHEIM (DE); PETER STAHL, BERNRIED (DE); WINFRIED ALBERT, EBERFING (DE); DOLORES SCHENDEL, MUNCHEN (DE); CHRISTIAN BOITARD, PARIS (FR); PETER VAN ENDERT, PARIS (FR); GUNTHER-GERHARD JUNG, TUBINGEN (DE)

Correspondence Address: Arent Fox Kintner Plotkin and Kahn, PLLC, 1050 Connecticut Ave. N.W., Suite 600, Washington, DC, 20036-5339, US

(21) Appl. No.: 08/981,824

(22) PCT Filed: Jul. 15, 1996
(86) PCT No.: PCT/EP96/03093

(30) Foreign Application Priority Data
Jul. 14, 1995 (DE) 195 25 784.7

(51) Int. Cl. A61K 38/00; A61K 39/00; C07K 17/00; C07K 16/00; C07K 7/00; C07K 5/00

(52) U.S. Cl. 424/185.1; 514/13; 530/324

(57) ABSTRACT

the invention concerns autoreactive peptides, peptide MHC complexes, t cell subpopulations which react therewith as well as diagnostic and therapeutic applications of these compounds.

### DESCRIPTION

The present invention concerns peptides which cause an autoimmune reaction, complexes of these peptides with molecules of the major histocompatibility complex (MHC), T cell subpopulations which react with the peptides or/and the complexes of peptides and MHC molecules as well as diagnostic and therapeutic applications of these compounds.

The elucidation of the molecular relationship in the development of autoimmune diseases such as rheumatoid arthritis and juvenile diabetes (IDDM) has progressed very rapidly in recent years and concrete applications for the early diagnosis and a causal therapy of these diseases is recognizable.

Today it is regarded as certain that environmental factors also play a role in the development of these diseases in addition to a genetic disposition. Of the level of genetic risk factors only a few alleles of the MHC class II antigens are closely associated with this disease for example in the case of IDDM. Thus it is possible to define a risk group for IDDM by analysing these alleles (cf. e.g. Thomson et al., Am. J. Hum. Genet. 43 (1988), 799-816 or Todd et al., Nature 329 (1987), 599-604).

Environmental factors involved in the development of IDDM are probably exogenous peptide sequences that acts as immunogen. Among others viral antigens which have partial homologies to endogeneous structures have been discussed in this connection. Under particular circumstances and in particular in the postnatal phase antigens taken up via the fool such as bovine serum albumin can induce an immune response which, due to homologies to endogeneous structures, can start an autoaggressive process.

Typical for the course of the disease in case of IDDM is the progressive destruction of pancreas β cells by cytotoxic lymphocytes. This process begins a long time before a recognizable disturbance of glucose metabolism. When the manifestation of diabetes is recognizable already over 90% of the β cells are destroyed. It would therefore be extremely important to detect these autoaggressive T cells at an eary stage in persons at risk in order to provide the affected individuals with a causal therapy.

Nowadays it is regarded as certain that the destruction of endogenous tissue in autoimmune diseases progresses very slowly at the start. In the initial stage of this process the autoaggressive T cells probably recognize only one or a few autoantigens. Publications by Kaufman et al. (Nature 368 (1993), 69-72) and Tisch et al. (Nature 368 (1993), 72-78) on an animal model (NOD mouse) for type I diabetes have shown that in the spontaneously occurring diabetes of this mouse strain the initial autoimmune reaction mediated by T cells is directed against glutamic acid decarboxylase. In this process only one to 2 epitopes of the C terminus of glutamic acid decarboxylase (GAD) are recognized initially in the NOD mouse. At this time no changes in the glucose metabolism can yet be determined—as described above—whereas in contrast a perinsulitis is already detectable. The spectrum of the peptides of GAD recognized by the autoaggressive T cells does not expand until later in the course of the disease. After the diabetes becomes manifest pre-activated T cells against other islet cell antigens are also detectable e.g. peripherin, heat shock protein HSP 65 and carboxypeptidase H.

There are indications that also in humans the immune response towards GAD is causally associated with the development of type I diabetes. Thus for example autoantibodies against GAD can be detected in over 80% pre-diabetics whereby the etiological role of these autoantibodies is, however, estimated to be low. Rather it is assumed that in the case of type I diabetes there is a progressive destruction of pancreas B cells by T lymphocytes. These T lymphocytes directed against GAD have already been detected by several research groups (Harrison et al., J. Clin. Invest. 89 (1992), 1161; Honeyman et al., J. Exp. Med. 177 (1993), 535). The autoantibodies found by these groups reacted with a peptide fragment of the GAD 67 kd molecule composed of amino acids 208 to 404.

Autoimmunely reacting polypeptides from the human GAD 65 kd molecule are disclosed in EP-A-0 519 469. These polypeptides have the amino sequence:

X-P-E-V-K-(T or E)-K-Z

in which X is an optional sequence selected from 1 to 10 amino acids and Z is an optional sequence selected from 1 to 8 amino acids.

Autoreactive peptide sequences from the human GAD 65 kd are proposed in the European Patent Application No. 95 100 764.0 comprising:

(a) the amino acid sequence

G-M-A-A-L-P-R-L-I-A-F-T-S-E-H-S-H-F-S-L-K-K-G-A-A,

(b) the amino acid sequence

E-R-G-K-M-I-P-S-D-L-E-R-R-I-L-E-A-K-Q-K,

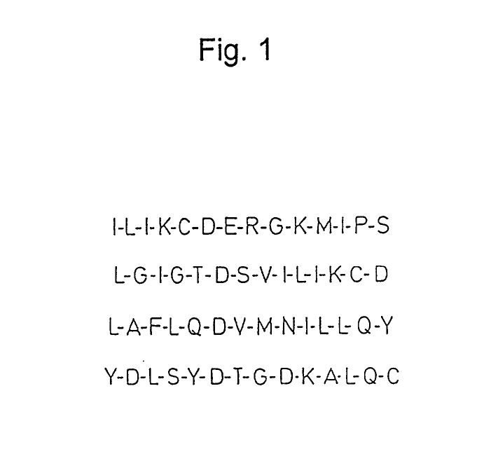

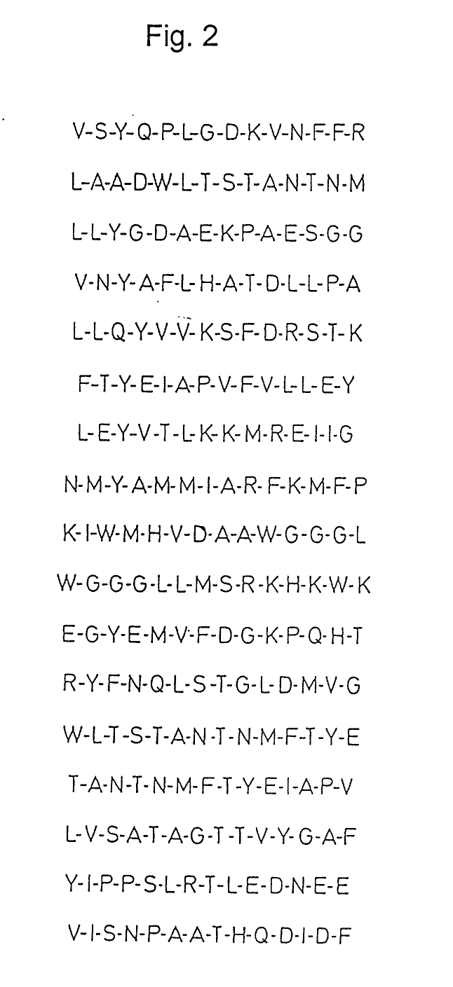

(c) one of the amino acid sequences shown in FIG. 1 or 2,

(d) partial regions of the amino acid sequences shown in (a), (b) or/and (c) with a length of at least 6 amino acids or/and

(e) amino acid sequences which have an essentially equivalent specificity or/and affinity of binding to MHC molecules as the amino acid sequences shown in (a), (b), (c) or/and (d).

An object of the present invention was to provide new autoreactive peptides that react with T cells from type I diabetics and especially with T cells from recently discovered type I diabetics and thus define early autoepitopes.

This object is achieved by peptides, peptide derivatives or molecules binding analogously which are suitable for the detection, isolation, multiplication, anergization or/and elimination of autoreactive T cells. One subject matter of the invention is thus a peptide or peptide derivative comprising:

(a) the amino acid sequence (I)

D-V-N-Y-A-F-L-H-A-T-D-L-L-P-A-C-D-G-E-R,

(b) the amino acid sequence (II)

S-N-M-Y-A-M-M-I-A-R-F-K-M-F-P-E-V-K-E-K,

(c) the amino acid sequence (III)

N-W-E-L-A-D-Q-P-Q-N-L-E-E-I-L-M-H-C-Q-T,

(d) the amino acid sequence (IV)

T-L-K-Y-A-I-K-T-G-H-P-R-Y-F-N-Q-L-S-T-G,

(e) the amino acid sequence (V)

P-R-Y-F-N-Q-L-S-T-G-L-D-M-V-G-L-A-A-D-W,

(f) the amino acid sequence (VI)

T-Y-E-I-A-P-V-F-V-L-L-E-Y-V-T-L-K-K-M-R,

(g) the amino acid sequence (VII)

F-F-R-M-V-I-S-N-P-A-A-T-H-Q-D-I-D-F-L-I,

(h) partial regions of the amino acid sequence shown in (a), (b), (c), (d), (e), (f) or/and (g) with a length of at least 6 amino acids or/and

(i) amino acid sequences which have an essentially equivalent specificity or/and affinity of binding to MHC molecules as the amino acid sequences shown in (a), (b), (c), (d), (e), (f), (g) or/and (h).

The amino acid sequences (I) to (VII) correspond to the amino acid residues 86-105 (I), 246-265 (II), 146-165 (III), 166-185 (IV), 176-195 (V), 206-225 (VI) and 556-575 of human GAD 65.

It was surprisingly found that peptides which correspond to amino acid sequences (I) to (VII) of human GAD 65 exhibited a specific reaction with T cell subpopulations which were isolated from recently discovered type I diabetics. Thus the peptide according to the invention are early autoepitopes which can be used for a very early diagnosis of type I diabetes. In addition the peptides according to the invention can also be used therapeutically by inactivating the T cell population that is reactive to the peptides.

A particularly preferred peptide is a partial peptide of peptide (VII) having the amino acid sequence SNPAATHQDIDFLI (VIIa) corresponding to the amino acid residues 562-575 of human GAD 65. By shortening analyses it was found that this peptide represents the minimal stimulatory sequence of the peptide (VII) especially with regard to its C-terminus. When it was shortened by only a single amino acid at the C-terminus (isoleucine) it was found that the peptide nearly completely lost its ability to stimulate.

Preferred examples of T cell subpopulations with which the peptides according to the invention of amino acid sequences (I) or/and (II) are the T cell lines R.B. and M.C. or T cells of an equivalent binding specificity.

The amino acid sequences (I) to (VII) are partial regions from the 65 kd isoform of human glutamic acid decarboxylase (GAD) the complete amino acid sequence of which has been described by Bu et al., (Proc. Natl. Acad. Sci. USA 89 (1992), 2115 ff.). The amino acid sequences (I) to (VII) were found by setting up T cell lines from the peripheral blood of type I diabetics and subsequently stimulating them in vitro with recombinant human GAD and testing these T cell lines in a proliferation assay with synthetic peptide sequences which were derived from the human GAD sequence.

The peptides according to the invention can be produced by known synthesis procedures by means of chemical methods or by genetic engineering by cloning and expressing a DNA sequence coding for this peptide in a suitable host cell in particular E. coli.

In addition the present invention also encompasses peptides with partial regions of the stated specific amino acid sequences (I), (II), (III), (IV), (V), (VI) or (VII) which have a length of at least 6 amino acids preferably of least 8 amino acids particularly preferably of at least 10 amino acids and most preferably of at least 15 amino acids. The minimum length of a peptide according to the invention is determined by its ability to recognize a MHC molecule, to bind specifically to it and to react with the corresponding T cell receptor.

The maximum length of the sections in a peptide according to the invention derived from GAD and binding to MHC is preferably 100 amino acids particularly preferably 50 amino acids and most preferably 25 amino acids.

In addition to peptides with the amino acid sequences (I) to (VII) or partial regions thereof the invention also concerns other peptides with amino acid sequences which have an essentially equivalent specificity or/and affinity of binding to MHC molecules as the aforementioned sequences and are preferably derived from the amino acid sequences (I) to (VII) by substitution, deletion or insertion of individual amino acid residues or short section of amino acid residues or modified substances bind analogously.

The present invention in particular also concerns peptide variants whose sequences do not completely correspond with the aforementioned amino acid sequences but which only have identical or closely related “anchor positions”. The term “anchor position” in this connection denotes an essential amino acid residue for binding to a MHC molecule in particular to a MHC molecule of the classes DR1, DR2, DR3, DR4 or DQ. The anchor position for the DRB1*0401 binding motif are for example stated in Hammer et al., Cell 74 (1993), 197-203. Such anchor positions are conserved in peptides according to the invention or are optionally replaced by amino acid residues with chemically very closely related side chains (e.g. alanine by valine, leucine by isoleucine and vica versa). The anchor position in the peptides according to the invention can be determined in a simple manner by testing variants of the aforementioned specific peptides for their binding ability to MHC molecules. Peptides according to the invention are characterized in that they have an essentially equivalent specificity or/and affinity of binding to MHC molecules as the aforementioned peptides. The peptides derived from peptides of amino acid sequences (I) to (VII) preferably have a sequence homology of at least 30% particularly preferably of at least 50% and most preferably of at least 60% to the starting peptides or partial sequences thereof.

Examples of variants of the specifically stated peptides are the corresponding homologous peptide sections from human GAD 67 the complete amino acid sequence of which has also been described by Bu et al., Supra.

The term “essentially equivalent specificity or/and affinity of binding to MHC molecules” also includes an improved binding specificity or/and affinity compared to the amino acid sequences (I) to (VII) which is especially found in the case of shortened peptides which has a length of preferably 8 to 15 amino acids.

In addition the present invention also encompasses peptide derivatives. This term includes peptides in which one or several amino acids have been derivatized by a chemical reaction. Examples of peptide derivatives according to the invention are in particular molecules in which the back-bone or/and reactive amino acid side groups e.g. free amino groups, free carboxyl groups or/and free hydroxyl groups have been derivatized. Specific examples of derivatives of amio groups are sulfonic acid or carboxylic acid amides, thiourethane derivatives and ammonium salts e.g. hydrochloride. Examples of carboxyl group deriviatives are salts, esters and amides. Examples of hydroxyl group derivatives are O-acyl or O-alkyl derivatives. The term peptide derivative according to the invention in addition also includes those peptides in which one or several amino acids have been replaced by naturally occurring or non-naturally occurring amino acid homologues of the 20 “standard” amino acids. Examples of such homologues are 4-hydroxyproline, 5-hydroxylysine, 3-methylhistidine, homoserine, ornithine, β-alanine and 4-amino butyric acid.

Those peptides are in particular preferred which have an essentially equivalent specificity or/and affinity of binding to MHC molecules such as peptides with the amino acid sequences (I) to (VII) but which in contrast to these peptides do not cause an activation of T cells but rather produce an anergic state in T cells.

Polypeptides are encompassed by the present invention in which the MHC-binding peptide section is a component of a larger polypeptide unit in which the compound of MHC-binding peptides and the rest of the polypeptide unit preferably have a pre-determined breaking point e.g. a protease cleavage site.

A further subject matter of the present invention is a peptide or peptide derivative which carries a signal generating substance or a marker group e.g. a fluorescent marker group (e.g. rhodamine, phycoerythrine), digoxin, biotin, a radioactive group or a toxine group (e.g. ricine, choleratoxine etc.). The peptide can be used as a diagnostic agent for in vivo or in vitro (e.g. imaging) applications or as a therapeutic agent by coupling the peptide according to the invention with marker groups. In addition the peptide according to the invention can also for example be present in a cyclized form or in an oligomeric form in which the important sequences for binding to the MHC molecule are separated from one another by spacer regions.

The invention also concerns peptide mimetic substances which have an essentially equivalent specificity or/and affinity of binding to MHC molecules as the aforementioned peptides or peptide derivatives. Peptide mimetic substances or peptide mimetics are compounds which can replace peptides in their interaction with MHC molecules as compared to the native peptides have an increased metabolic stability, improved bioavailability and longer duration of action. Methods for the production of peptide mimetics are described by Giannis and Kolter, “Angew. Chem.” 105 (1993), 1303-1326, Lee et al., Bull. Chem. Soc. Jpn. 66 (1993), 2006-2010 and Dorsch et al., “Kontakte” (Darmstadt) (1993) (2), 48-56. Reference is made to the disclosure of these literature references with regard to the synthesis of peptide mimetic substances according to the invention.

A further subject matter of the present invention is a complex which includes at least one peptide according to the invention, peptide derivative or peptide mimetic and at least one MHC molecule or peptide-binding derivative of a MHC molecule. In this complex a peptide, peptide derivative or peptide mimetic with a binding constant of at least 10⁻⁷ /mol particularly preferably in the range of 10⁻⁸-10⁻⁹ l/mol is bound to a MHC molecule or a peptide-binding derivative of a MHC molecule. Alternatively the peptide, peptide derivative or peptide mimetic can also be covalently be coupled to the MHC molecule e.g. by means of a photolinker or as a covalent genetic peptide-MHC fusion. Such a peptide-MHC fusion protein preferably contains a HLA-DR beta chain and an autoreactive peptide genetically fused to it. The complex particularly preferably contains a MHC class II molecule or a peptide-binding derivative thereof.

The MHC class II molecule is preferably of the DR type for example of the DR1, DR2, DR4 or DQ6 type. The MHC class II molecule is preferably of the DR1 type (subtype DRB1*0101), DR2 (subtype B1*1501, DR B1*1502, DR b1*1601 or Dr B5*0101), DR4 (subtype DR B1*0401) or DQ6 (subtype DQ B1*0602). The T cell line R.B. proliferates with the autoreactive peptide of amino acid sequence 86-105 of GAD 65 kd in the presence of the DR B1 allele 0101. The T cell line M.C. proliferates with the autoreactive peptide of amino acid sequence 246-265 of rGAD in the presence of the DR B1 allele 1501 or/and of the DQ B1 allele 0602. The DR B1 allele 0401 was identified as a restriction element for the autoreactive peptide with the amino acid sequence 556-575 of GAD.

The nucleotide sequences of genes coding for a MHC class molecule of the above subtype are published in Corell et al., (Mol. Immunol. 28 (1991), 533-543). Reference is hereby made to the content of this publication.

The term “peptide-binding derivative of a MHC molecule” includes fragments of MHC molecules which are produced by proteolytic cleavage of native MHC molecules or by recombinant DNA techniques and which have essentially retained their peptide-binding properties. This term is also to be understood to include fusion proteins which have yet a further polypeptide component in addition to the MHC part responsible for peptide binding.

The peptide-MHC complexes according to the invention are preferably produced by association of peptide-free MHC molecules or MHC molecule derivatives with the peptides, peptide derivatives or peptide mimetics according to the invention. The production of peptide-free MHC molecules can for example be carried by unfolding native MHC molecules in order to dissociate bound peptides and refolding the empty MHC molecules (see Dornmair and McConnell, Proc. Natl. Acad. Sci. USA 87 (1990), 4134-4138 and WO91/14701).

On the other hand peptide-free MHC molecules can also be obtained by the recombinant production of MHC molecules or derivatives thereof. Examples of this are the expression of MHC class II molecules in fibroblasts (Germain and Malissen, Ann. Rev. Immunol. 4 (1990), 281-315) and the expression of soluble MHC class II molecule derivatives without membrane anchors in CHO cells (Wettstein et al., J. Exp. Med. 174 (1991), 219-228, Buelow et al., Eur. J. Immunol. 23 (1990), 69-76) and by means of the baculovirus expression system in insect cells (Stern and Wiley, Cell 68 (1992), 465-477; Scheirle et al., J. Immunol. 149 (1992), 1994-1999). MHC class I molecules have also been expressed in CHO cells (Fahnestock et al., Science 258 (1992), 1658-1662) in insect cells (Jackson et al., Proc. Natl. Acad. Sci. USA 89 (1992), 12117-12120; Matsamura et al., J. Biol. Chem. 267 (1992), 23589-23595) and in fibroblasts (Mage et al., Proc. Natl. Acad. Sci. USA 89 (1992), 10658-10661).

The expression of peptide-free MHC molecules is also known in E. coli (Parker et al., Mol. Immunol. 29 (1992), 371-378; Zhang et al., Proc. Natl. Acad. Sci. USA 89 (1992), 8403-8407; Garboczi et al., Proc. Natl. Acad. Sci. USA 89 (1992), 3429-3433; Altman et al., Proc. Natl. Acad. Sci. USA 90 (1993), 10330-10334). Reference is made to the technique for the recombinant expression of MHC molecules or MHC molecule derivatives described in these publications.

The MHC component of the complex according to the invention is preferably a recombinant MHC molecule or a peptide-binding derivative thereof and particularly preferably a soluble MHC molecule derivative in which the membrane anchor is partially or completely deleted.

In order to identify MHC molecules which present the autoreactive peptide according to the invention the antigen presenting cells of a donor are incubated with the peptide according to the invention in a labelled form in which bound peptides are preferably firstly associated by denaturing native MHC molecules. Subsequently the labelled MHC-peptide complexes can be immunoprecipitated with subtype-specific antibodies which are directed against frame work-specific determinants of the MHC molecules and are identified by the presence of the labelled peptides.

Alternatively EBV (Epstein-Barr virus) transformed B cells of the donor can be used as the antigen presenting cells.

The complexes according to the invention comprising a recombinant MHC molecule derivative can for example be produced by isolating DNA fragments for the soluble parts of the α and β chains of a MHC molecule e.g. a MHC-DR1, DR2 or DQ1 molecule by PCR in which cDNA from an EBV-transformed B cell line of a donor is used as a template which expresses the corresponding MHC molecule. In this step a purification aid e.g. an oligohistidine segment (e.g. a hexahistidine segment) is preferably introduced at the C terminus of the α and the β chain by appropriate selection of the PCR primer. The PCR products can be subsequently subcloned in E. coli and expressed as inclusion bodies. The inclusion bodies can be solubilized by known methods (cf. literature references for the expression of MHC molecules in E. coli, supra) and the MHC proteins can be purified by means of metal chelate affinity chromatography. Subsequently the α and β subunits are renatured in the presence of the peptide.

The peptide-MHC complex according to the invention can also carry a marker group as described above in which the marker group can be bound to the peptide component as well as to the MHC component of the complex by known methods.

A further subject matter of the present invention is an oligomerized peptide-MHC complex which contains at least 2 MHC molecules or MHC molecule derivatives which are associated by means of covalent or non-covalent interactions. Such an oligomerized peptide-MHC complex has the advantage over known (with regard to the MHC molecule) monomeric complexes of a higher affinity and thus an improved diagnostic or/and therapeutic efficacy.

In one embodiment of the present invention such an oligomerized complex can be produced according to known methods by covalent cross-linking of monomeric peptide/MHC molecule complexes by means of chemical coupling reagents e.g. N-succinimicyl-3(2-pyridylthio)propionate, 3-maleimidobenzoyl-N-hydroxysuccinimide ester, maleimidohexanoyl-N-hydroxy-succinimide ester, bis(maleimidomethyl)ether, dissuccinimidylsuberate, glutardialdehyde etc. Optionally individual amino acids of the peptide component or the MHC component can also be modified in such a way that special coupling reagents preferably attack at this site. Thus the introduction of additional cysteine or lysine residues by recombinant means in the protein component or by chemical synthesis in the case of the peptide component allows coupling via SH linkers or via amino groups.

In a further embodiment of the present invention the oligomerized peptide-MHC complex can be produced in such a way that the peptide component binding to the MHC molecule is used as an oligomer i.e. as a peptide molecule which contains at least 2 MHC-binding regions in which the sequences that are important for binding to the MHC molecules are separated from one another by spacer regions. These spacer regions are usually composed of 10-15 amino acids. One uses small hydrophilic amino acids e.g. glycine, alanine, serine, proline or combinations thereof. When peptide-free MHC molecules are renatured in the presence of these peptide oligomers these oligomerized complex according to the invention forms which contains the MHC molecules cross-linked by the oligomerized peptide component via non-covalent interaction.

In addition oligomerized peptide-MHC complexes can be produced by modification of MHC molecules produced by recombinant means. Thus during the construction of vectors for the expression of recombinant α or β chains of MHC class II molecules a gene segment can be cloned in preferably at the C terminus in each case which codes for an epitope that is recognized by an antibody. This antibody can be of the IgG type but preferably of the IgM type. The renatured monomeric peptide/MHC complexes are then incubated with an antibody that recognizes the introduced epitope so that non-covalently cross-linked immune complexes composed of several antibodies and several peptide-MHC complexes are produced. The introduction of DNA segments which code for an epitope into the DNA fragment coding for the α or β chain of the MHC molecule can be carried out by means of known molecular biological techniques e.g. by insertion into restriction sites or by site-directed mutagenesis.

The oligomerized peptide-MHC complex according to the invention contains a peptide which comprises the amino acid sequences (I), (II), (III), (IV), (V), (VI), (VII) or partial regions thereof or/and amino acid sequences derived therefrom or a peptide derivative or peptide mimetic thereof. The oligomerized complex can preferably be used as a diagnostic or therapeutic reagent for type I diabetes.

Thus the invention also concerns a pharmaceutical composition which contains a peptide, peptide derivative, peptide mimetic or/and a peptide-MHC complex as the active component optionally in combination with common pharmaceutical additives. The composition can in addition contain an accessory stimulating component e.g. cytokines such as IL-2 and IL-4 or/and the surface antigen B7 (Wyss-Coray et al., Eur. J. Immunol. 23 (1993), 2175-2180; Freeman et al., Science 262 (1993), 909-911) which can bind with the surface molecule CD-28 on a T cell. The presence of the accessory stimulating component has improved or/and modified the therapeutic action of the composition.

An additional subject matter of the present invention is the use of a pharmaceutical composition which contains a peptide, peptide derivative, peptide mimetic or/and peptide-MHC complex for the production of an agent for the diagnosis of diseases or a predisposition for diseases which influence the immune system or for the diagnosis of tumour diseases or a predisposition of tumour diseases in particular for the diagnosis of autoimmune diseases or a predisposition of autoimmune diseases e.g. diabetes type I or type II preferably diabetes type I.

Analogous diagnostic applications are, however, also possible for other autoimmune diseases. Examples of such autoimmune diseases are multiple sclerosis where reactive T cells against metylin basic protein or the proteolipid protein can be determined, rheumatoid arthritis where reactive T cells against collagen type II, cytokeratine and Hsp 65 can be determined, Basedow disease where reactive T cells against thyroid peroxidase can be determined.

In general a diagnostic application is possible for all diseases which influence the immune system such as e.g. also in the case of artheriosclerosis. In this case the disease has been found to be associated with an immune response against the heat shock protein Hsp 65 (Xu et al., Lancet 341, 8840 (1993), 255-259).

A further application is the diagnostic detection of T cells which react to tumour antigens. Examples of this are T cells against a melanoma-associated antigen MAGE 1 which has been isolated from melanoma patients (van der Bruggen et al., Science 254 (1991), 1643-1647). oligomerized complexes according to the invention can be used to already detect these T cells in a stage in which the tumour is not yet detectable by conventional methods due to a still too low a cell mass. In addition the detection of specifically reacting T cells can also be used to monitor an anti-tumour vaccination.

Hence a further subject matter of the present invention is a method for determining a specific T cell subpopulation which is characterized in that a sample containing T cells which is preferably derived from a body fluid e.g. whole blood is contacted with a peptide, peptide derivative, peptide mimetic or/and a complex according to the invention and the reaction of T cells with the peptide or complex is determined. A specific reaction of T cells with the complex or the peptide can then for example be detected by an increased T cell proliferation which can be measured by the incorporation of radioactivity. On the other hand the reaction of T cells can also be determined directly by using a labelled peptide or complex. In this embodiment the peptide or complex is preferably used with a fluorescent labelled group coupled thereto. The evaluation can for example be carried out FACS analysisin which the T cells are coupled with a first fluorescent label which is coupled to a T cell-specific antibody and then with the peptide-MHC complex which is coupled to a second fluorescent label and the presence of double-labelled cells is determined by fluorographic analysis. In this manner a T cell subpopulation is determined which is characterized by its reactivity with a peptide or peptide derivative according to the invention or/and with a peptide-MHC complex according to the invention. Due to the low concentration of the specific T cell population in blood a selection for pre-activated T cells e.g. a selective enrichment of IL-2 receptor-positive T cells is preferably carried out as the first step of the procedure by incubation with IL-2 or/and by incubation with IL-2 receptor antibodies and subsequent separation of the antibody-binding cells for example with immune magnetic methods. On the other hand the selection for pre-activated cells can be first carried out after contact of the T cells with the peptide or the complex.

In a modification of this method it is also possible to determine the ratio of pre-activated autoreactive T cells i.e. T cells with the IL-2 receptor as a surface marker to non-activated autoreactive T cells i.e. T cells without the IL-2 receptor.

This method can be used especially to diagnose type I diabetes but also for other diseases which influence the immune system or for the diagnosis of a predisposition for such diseases.

A further subject matter of the present invention is the use of a pharmaceutical composition which contains a peptide, peptide derivative, peptide mimetic or/and a peptide-MHC complex for the production of an agent for the treatment or prevention of diseases which influence the immune system. For the therapeutic application of the peptides according to the invention or the peptide-MHC complex according to the invention it is for example possible to use peptides or peptide-MHC complexes coupled to toxins and on the other hand it is also possible to use peptides alone or as components of the complex which although enabling and binding to the T cell receptor do not cause an activation of the T cell i.e. have an anergizing effect.

The therapeutic action of such anergizing peptide analogues is based on the fact that the T cell receptor (TCR) must interact with a peptide which is presented by a MHC antigen of class I or class II in order to activated the T cell. In this connection amino acids in anchor positions of the peptide are in particular responsible for the binding to the MHC molecule whereas other amino acids in the peptide contribute to the interaction with TCR and thus cause a T cell stimulation. Peptide analogues can thus be produced by amino acid substitution in the peptides which, due to the presence of the anchor positions, still bind to the MHC molecule but on the other hand only cause a partial or no T cell activation (cf. e.g. Sloan-Lancaster et al., Nature 363 (1993), 156-159). Such peptide analogues can for example have the effect that the expression of particular surface molecules is up-regulated (e.g. IL-2 receptor, LFA-1) but that no proliferation or cytokine expression occurs. T cells which interact with such a peptide analogue pass into a so-called anergic state i.e. they can no longer proliferated even as a result of a subsequent regular stimulation with an-immunogenic peptide. This anergic state lasts for at least 7 days and can therefore be used therapeutically in the treatment of an autoimmune disease.

A further therapeutic aspect of the present invention is that the peptide or the complex of peptide and MHC molecule can be used as an antigen. Such an antigen can in this case act as an immunogen i.e. as an agent stimulating the immune response or as a tolerogen i.e. as an agent which causes an immune response. The use as an immunogen can for example be applied in the vaccination against tumour antigens. Instead of the whole tumour cells previously used for this purpose it is possible to inject tumour-specific peptides recognized by the T cells in a complex with the appropriate MHC molecule in particular in the form of an oligomerized complex in order to induce a T cell response against the tumour. In order to increase the immune stimulation this complex can also be administered in combination with additional stimulating substances. Cytokines such as IL2 or IL4 are for example suitable for this purpose which are optionally and preferably covalently linked to the peptide-MHC complex according to the invention. A further possibility is to associate the complex with accessory components for T cell activation in particular with surface molecules that are essential for antigen presenting cells e.g. the surface molecule B7.

A preferred therapeutic formulation is to incorporate the MHC molecules loaded with peptide into artificial vesicles e.g. lipid vesicles which can optionally carry further membrane-bound molecules such as B7 or/and immobilized cytokines.

A further subject matter of the present invention is the isolation of T cell subpopulations which react with a peptide or peptide-MHC complex according to the invention. In such a method a sample containing T cells which is for example derived from a body fluid which has previously been taken from a patient is contacted with a peptide according to the invention or a peptide-MHC complex according to the invention, the T cells reacting with the peptide or complex are identified and they are optionally separated from other T cells. Also in this case a selection for pre-activated T cells i.e. T cells with the IL2 receptor can preferably be carried out before or/and after contact of the T cells with the peptide or the complex.

In such a process the peptide or the peptide-MHC complex can be used in an immobilized form on a support which synthesize the separation of the positively-reacting T cell population from other T cells. T cell lines can be set-up from the T cell subpopulation isolated in this manner by restimulation. These autoreactive T cell lines can then be used to immunize patients.

A specific immune therapy of type I diabetes comprises firstly the isolation of specific T cell lines against an autoantigen e.g. GAD 65 from IDDM patients. Then the fine specificity of the T cell lines is determined i.e. the autoreactive peptides are identified. Those T cell lines are selected for the later inoculation of the patients which recognize a predominant peptide i.e. a peptide against which several of the isolate T cell lines react. In particular these are T cell lines which recognize the amino acid sequences (I), (II), (III), (IV), (V), (VI) or (VII).

If no unequivocal predominant peptide can be found in the patient, several T cell lines have to be mixed for the later inoculation. The selected T cell clones are stimulated again before the inoculation with antigen-presenting cells and the corresponding peptides in order to ensure a good expression of activation molecules and in particular the T cell receptors. Then the T cell lines are inactivated e.g. by heat treatment or/and radioactive irradiation preferably in a dose in the range of 4000-10000 rad particularly preferably ca. 8000 rad and injected subcutaneously into the patient from which they were obtained using a cell number of preferably 10⁷ to 5×10⁷. Usually at least three injections are distributed over a period of 6 to 12 months.

Subsequently one can test the T cell response of the patient to the inoculate. For this purpose the peripheral blood lymphocytes (PBLs) of the patient are isolated e.g. by means of Ficoll density gradient centrifugation and the proliferation caused by the inoculate is tested in the standard proliferation test. If the immunization has proceeded successfully there should be a clearly detectable proliferation of the patient's PBLs towards the inoculate. A further control of the success of the immunization can be carried out be determining the frequencies of the GAD-reactive T cells of the patient during the course of the immunization. this can for example be carried out by the standard method of limiting dilution using autologous stimulator cells which have been irradiated with e.g. 4000 rad after incubation with GAD. If the immunization has proceeded successfully the frequency of autoreactive T cells decreases considerably.

After further narrowing down the surface structures on the T cells of the inoculate that are recognized by the regulatory T cells it is then also possible to immunize with partial structures of the regulatory T cells e.g. with segments of the T cell receptor.

On the other hand T cells capable of dividing can be reinjected in the case of an anti-tumour vaccination which can lead to an active immunization of the patient against tumour cells.

In the diagnostic and therapeutic methods for identifying or activating/inhibiting specific T cell subpopulations an anti-idiotypic antibody can be used instead of the peptides or peptide-MHC molecules according to the invention which simulate the action of the MHC peptide complex. Such antibodies can be easily obtained by using a specific T cell subpopulation against a particular peptide as the immunogen to produce an antibody (e.g. in a mouse) or by firstly producing a first antibody against the MHC peptide complex and then an anti-idiotypic antibody against the first antibody.

Thus a subject matter of the present invention is also an antibody (first antibody) against a peptide or peptide derivative according to the invention or against a complex according to the invention obtainable by immunization with the peptide, peptide derivative or complex according to the invention and isolating an antibody produced by immunization preferably a monoclonal antibody produced by the method of Köhler and Milstein or further development thereof.

Finally the invention also concerns an anti-idiotypic antibody against the first antibody obtainable by immunization with the first antibody which is directed against the peptide or peptide derivative or the complex and isolation of an anti-idiotypic antibody obtianed by immunization.

Yet a further subject matter of the present invention is a T cell which reacts with an autoreactive peptide, peptide derivative or peptide mimetic or a complex of peptide and MHC molecule according to the invention. Preferred examples are T cells which are derived from the T cell lines R.B., M.C., 24/31 or 40/2 or have an equivalent T cell receptor binding specificity i.e. recognize a peptide presented by a MHC molecule or a peptide derivative having the amino acid sequences (I), (II), (III), (IV), (V), (VI) or/and (VII) or/and partial regions of these amino acid sequences. The T cell line <GAD> 40/2 has been deposited on the 10.07.1996 at the “Deutsche Sammlung für Mikroorganismen und Zellkulturen (DSMZ)”, Mascheroder Weg 1b, D 38124 Braunschweig according to the rules of the Budapest Treatyunder the reference No. DSM ACC 2278. A confirmation of receipt by the depository office is attached to the application documents.

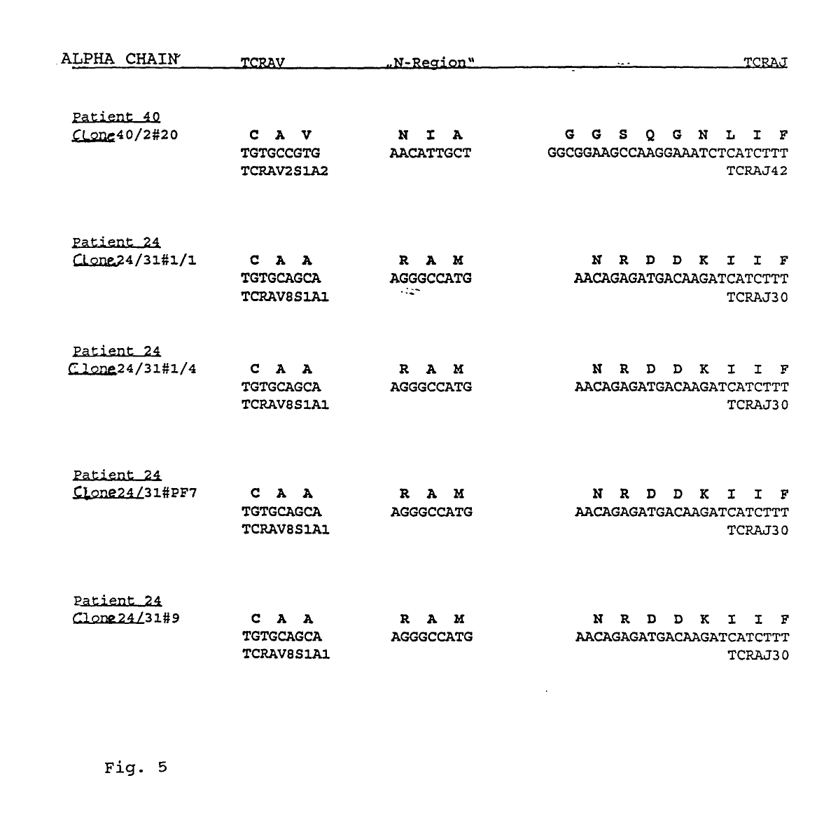

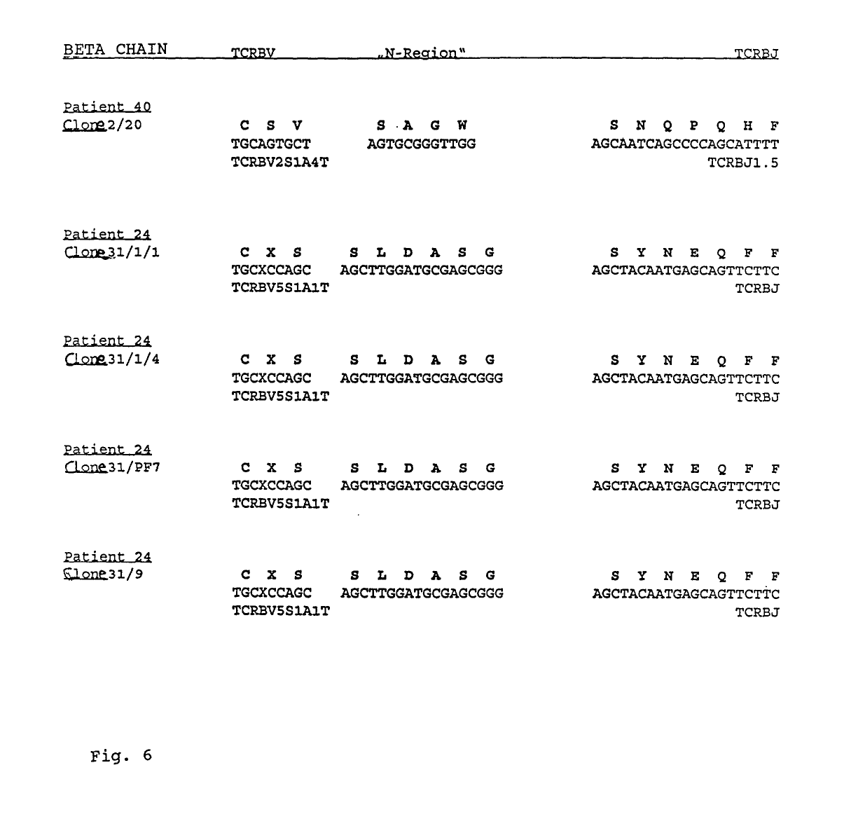

Examples of preferred T cells have a T cell receptor which comprises a TCR ∝ chain containing one cf the CDR3 regions shown in FIG. 5 or/and a TCR β chain containing one of the CDR 3 regions shown in FIG. 6. The invention also concerns T cell receptors which have amino acid sequences that are at least 70% homologous, preferably at least 80% homologous and particularly preferably at least 90% homologous to the CDR3 regions shown in FIG. 5 or 6.

Yet a further subject matter of the present invention is a polypeptide having T cell receptor activity which binds to an inventive peptide, peptide derivative, peptide mimetic or to a MHC complex containing one of these. A polypeptide according to the invention preferably comprises a TCR a chain containing one of the CDR3 regions shown in FIG. 5 or an amino acid sequence that is at least 70% homologous thereto or/and a TCR β chain containing one of the CDR3 regions shown in FIG. 6 or an amino acid sequence that is at least 70% homologous thereto.

Finally the present invention also concerns the use of peptides of GAD, in particular human GAD 65, peptide derivatives derived therefrom or peptide mimetics for the production of a pharmaceutical agent which leads to the formation of an immune tolerance when administered to diabetes patients. Peptides having the amino acid sequences (I), (II), (III), (IV), (V), (VI), (VII) or amino acid sequences proposed in EP 95 100 764.0, partial regions of these peptides with a length of at least 6 amino acids or/and amino acids with an essentially equivalent specificity or/and affinity of binding MHC molecules as the aforementioned peptide sequences are preferably used for this. The peptides preferably have a length of at least 8 amino acids particularly preferably a length of 10 to 25 amino acids.

The basis of this invention are observations that were made during the in vitro use of peptides for T cell stimulation. Thus if already established T cell lines are stimulated with a peptide that has been identified as being reactive e.g. a peptide with a length of 20 amino acids, then a proliferation occurs which is almost as high as when using the native antigen e.g. recombinant human GAD 65 kd. When the T cells that are expanded in this manner are again restimulated in a second cycle after ca. 10 days a much weaker proliferative response is obtained than if the native antigen is used in the first cycle. This finding is independent of whether the peptide or the native antigen is used again in the second cycle. A third restimulation usually ends in a complete dying of the T cells even if native GAD 65 kd is used as the antigen.

For this form of application the peptides are administered in relatively high doses preferably of 1 to 100 mg particularly preferably of 3 to 30 mg and most preferably of 5 to 10 mg per kg body weight.

In addition it is preferred that after the first administration of the peptides i.e. the first vaccination, at least a further second vaccination and particularly preferably at least a third vaccination is carried out. In the second and subsequent optional vaccinations the peptides, complete GAD or/and a part thereof containing the sequence of the peptide that were already used for the first vaccination are preferably used. In the case of a multiple vaccination the intervals between the individual vaccinations are preferably 5 to 25 days and particularly preferably 7 to 14 days.

In addition it is intended to elucidate the invention by the following examples in conjunction with the FIGS. 1, 2 3A, 3B, 3C, 4A, 4B, 5 and 6 and the sequence protocols SEQ ID NO. 1 to 30.

FIG. 1 shows autoreactive amino acid sequences according to EP 95 100 764.0,

FIG. 2 shows further autoreactive amino acid sequences according to EP 95 100 764.0,

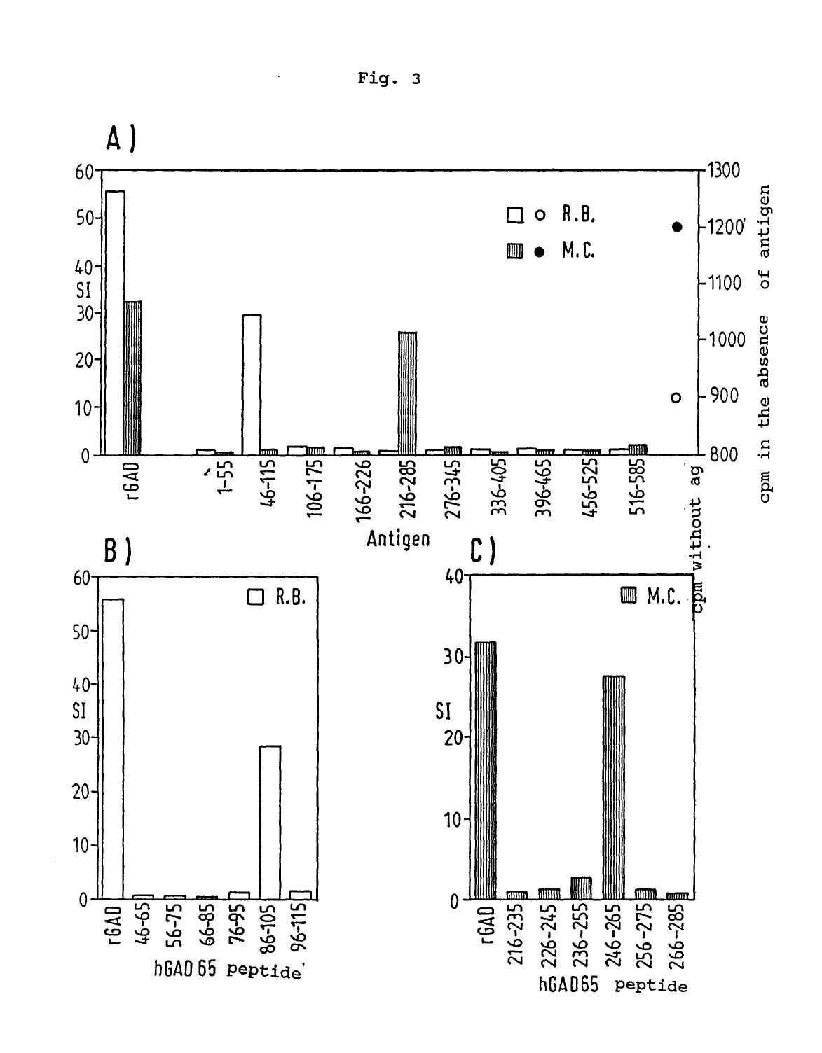

FIG. 3A shows the result of a peptide screening assay of the T cell lines R.B. and M.C. using recombinant GAD and peptide pool,

FIG. 3B shows the result of a proliferation assay of the T cell line R.B. with individual peptides from rGAD,

FIG. 3C shows the result of a proliferation assay of the T cell line M.C. with individual peptides from rGAD,

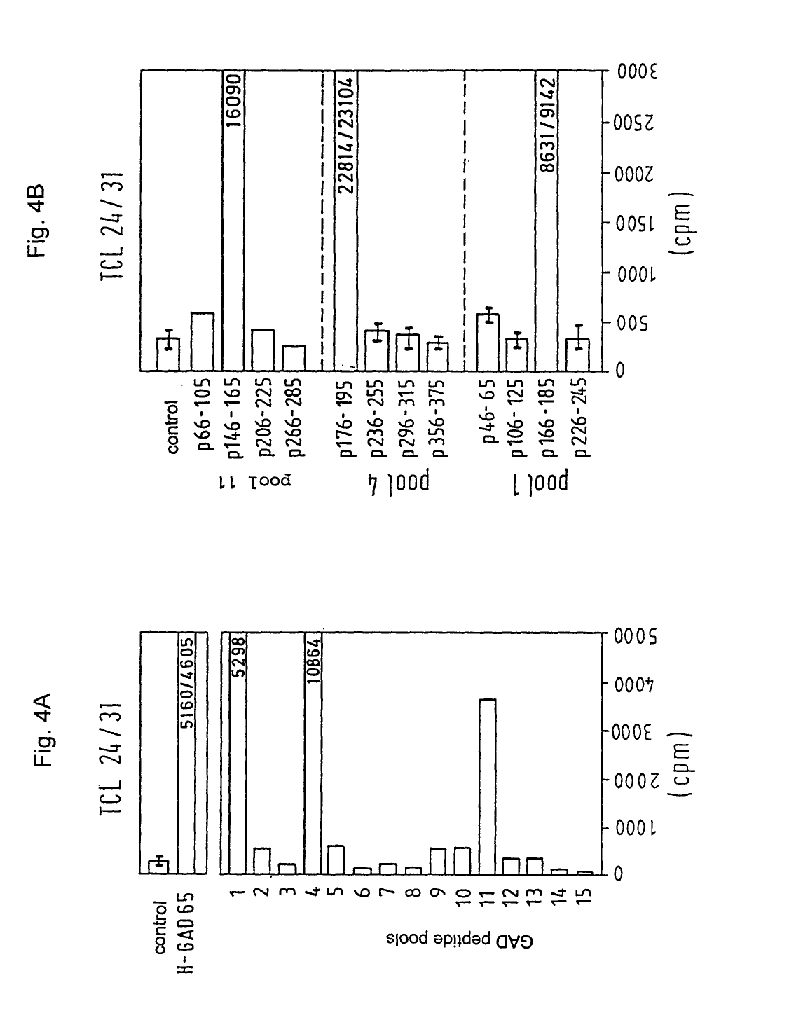

FIG. 4A shows the result of a peptide screening assay of the T cell line 24/31 using recombinant human GAD or peptide pools,

FIG. 4B shows the result of a proliferation assay of the T cell line 24/31 using individual peptides from GAD,

FIG. 5 shows the result of sequencing TCR α chains from clones of the T cell lines 40/2 and 24/31,

FIG. 6 shows the result of sequencing TCR β chains from clones of the T cell lines 40/2 and 24/31.

SEQ ID NO. 1-7 show the autoreactive amino acid sequences (I)-(VII) according to the invention

SEQ ID NO. 8-11 show the autoreactive amino acid sequences according to FIG. 1,

SEQ ID NO. 12-28 show the autoreactive amino acid sequences according to FIG. 2 and

SEQ ID NO. 29-30 show further autoreactive amino acid sequences according to EP 95 100 764.0.

### EXAMPLE 1

### Establishing GAD-specific T Cell Lines

1. Primary Stimulation

The peripheral blood lymphocytes (PBLs) are isolated by ficoll density gradient centrifugation from EDTA blood of type I diabetics. The cells are washed twice in RPMI medium and then taken up in a culture medium composed of RPMI 1640, 5% human serum, 2 mM glutamine and 100 U/ml penicillin and 100 μg/ml streptomycine. 100 μl cell suspension corresponding to 100,000 cells is sown per well of a 96 well round-bottom plate. Subsequently recombinant human GAD 65 kd (rGAD) is added which has been expressed in a baculovirus system at a final concentration of 3 to 5 μg/ml. The cells are incubated for 3-4 days in an incubator at 37° C./7% CO₂. After this period 100 μl IL-2 (5 U/ml) is added. After a further 3-4 days 100 μl is aspirated from all culture preparations and again 100 μl Il-2 (5 U/ml) is added. This is repeated every 3-4 days.

2.Restimulation

The first restimulation is carried out on the 14th day after the start of the primary stimulation. In comparison to the primary stimulation twice the number of autologous PBLs are isolated by means of ficoll and adjusted to a cell concentration of 2×10⁶/ml for this. One half of these stimulator cells is incubated with the antigen rGAD (final concentration 3 to 5 μg/ml) for 2 hours/37° C./7% CO₂. The other half is incubated only with culture medium without antigen under the same conditions. Subsequently all stimulator cells are irradiated with 4000 rad. The stilumator cells are then distributed in 96 well round-bottom plates (each time 100,000 cells/well) and such that always one well containing stilulator cells containing antigen is adjacent to a well with stimulator cells without antigen.

Subsequently the T cells are prepared from the primary stimulation preparations. For this purpose the supernatant of the primary stimulation mixtures are aspirated and the cells are washed twice in the plates with 100 μl wash medium in each case (Dulbeccos modified eagle medium=DMEM). Between washes the cells in the plates are centrifuged at 400 g. Subsequently the cells are taken up in 100 μl culture medium in each case and 50 μl of each is distributed into two adjacent wells of the restimulation plate. In this way the T cells in one well are incubated with antigen and in the adjacent well without antigen the antigen-specificity of the restimulation can be controlled.

After the 2nd or 3rd day after the beginning of the restimulation it is possible to microscopically assess the proliferation. In this case only those microculture pairs are regarded as relevant in which proliferation occurs only in the well in which antigen is present. From the 4th day onwards 100 μl IL-2 (5 U/ml) is again added to each culture well. Up to the 14th day ca. 50% of the culture medium is replaced by IL-2 (5 U/ml) every 3-4 days.

If growth is good the cultures are divided onto several 96 well plates. If the restimulation is later they can be divided into larger wells. A restimulation is carried out every 2 weeks by the aforementioned method. From the 3rd restimulation onwards the specificity of the microcultures is determined in a proliferation test.

3. Proliferation Test using Recombinant Human GAD 65 kd

All tests are carried out in at least double preparations.

a) Stimulator Cells:

Autologous PBLs or PBLs with identical HLA class II antigens of a normal donor are used as stimulator cells (APC). The PBLs are divided in a number of 100,000 per well of a 96 well plate and admixed with rGAD at a final concentration of 3 to 5 μg/ml. In control preparations an equal volume of medium is placed in the wells instead of antigen. After incubating for 2 hours at 37° C. and 7% CO₂ the stimulator cells are irradiated with 4000 rad.

b) T cells

The T cells used are always derived from the final phase of a restimulation period. They are washed three times with DMEM to free them of antigen and IL-2 and 6000 to 10,000 cells are distributed per 96 well.

After 3-4 days at 37° C./7% CO₂ 1 μCi 3H-thymidine was added and it was incubated for a further 16-20 hours. Afterwards the cells are transferred onto a glass fibre filter using a cell harvester instrument and the incorporated radioactivity is determined in a B counter instrument. The proliferation activity of the T cell lines is expressed by a stimulation index (SI). This is the quotient of the cpm in the presence of rGAD divided by the cpm in the control preparations without antigen. FIG. 3A (column rGAD) shows a typical result of a proliferation test using rGAD and the lines R.B. and M.C.

4. Proliferation Test using peptides which are Derived from the H-GAD 65 kd Sequence

T cell lines which had been expanded over at least 4 restimulation cycles and which reacted with rGAD in the proliferation test were additionally tested with overlapping peptides of rGAD. The object of these experiments is to define the epitopes of rGAD recognized by the T cells. For this overlapping 20 mer peptide of rGAD are firstly synthesized (overlapping region 10 amino acids, a total of 59 different peptides).

In each case 4-5 of these peptides are combined to a pool and added to the stimulator cells at a final concentration of 5 μg/ml (preparation of stimulator cells as described in section 3a).

Afterwards 6000-20,000 T cells are added per microculture well. The subsequent procedure is analogous to that described in section 3b.

FIG. 3A shows the results of this peptide screening assay. The T cell line R.B. reacts with the peptide pool which contains the rGAD sequence section 46-115 whereas the T cell line M.C. recognizes the sequence section 216 -285. The reactivities of the T cell lines R.B and M.C. with the individual peptides of the respective peptide pool are shown in FIGS. 3B and 3C. The line R.B. reacts exclusively with the peptide p86-105 whereas the line M.C. is specific for the peptide p246-265. In these proliferation tests the peptides were used at a concentration of 3 μg/ml.

FIG. 4A shows the result of a further peptide screening test using the T cell line 24/31. This T cell line reacts specifically with the peptide pool 1, 4 and 11. The reactivities of this T cell line with the individual peptides from these pools is shown in FIG. 4B. From this it can be seen that the T cell line 24/31 reacts with the peptides p166-185 and p176-195.

### EXAMPLE 2

### Determination of the Subtype of MHC Molecules which Present the T Cell Line R.B. and M.C. Autoreactive Peptides

The experimental procedure is carried out analogous to example 1.4. However, no autologous PBLs were used as antigen-presenting cells but rather Epstein Barr virus transformed B cells with defined MHC allels (so-called homozygote typing cell lines). These were selected such that there is only a partial correspondence with the MHC class II molecules of the donor of the T-cell lines e.g. identity with regard to the DR allels, non identity with regard to the DQ allels. In a departure from the described example 1.4 the peptides were washed out after the antigen pulse in order to avoid an autopresentation by the T cells.

The results of this test are shown in Table 1. The T cell proliferation is expressed as a stimulation index (SI).

The result of this analysis is unequivocal in the case of the T cell line R.B. Only when the antigen-presenting cells present the peptide p86-106 in association with DRB1*0101, is there a stimulation of the T cells. Other DR allels cannot present the peptide and involvement of the DQ allel DQB1*0501 can be excluded (see result with the antigen-presenting cells MZ070782). Thus DRB1*0101 is the restriction element for the T cell line R.B. The restriction element for the T cell line M.C. could not be elucidated in detail by this type of analysis since DR allel DRB1*0501 and the DQ allel DQB1*0602 are not present closely coupled in the Caucasian population. The analysis resulted in the presentation of the peptide either via the DR allele DRB1*0501 or 1601 or via the DQB1*0602 allel.

TABLE 1

| | | | T cell line proliferation | | |
|---|---|---|---|---|---|
| | | | | peptide | |
| | | no antigen | rGAD | 86-105 | 246-265 |
| APC | DRB1*:DQB1* | (CPM) | (SI) | (SI) | (SI) |
| | R.B. T cell line | | | | |
| PBMC | 0101/0401:0501/0302 | 898 | 56 | 28 | |
| JESTHOM | 0101:0501 | 840 | — | 20 | |
| HOM2 | 0101:0501 | 215 | — | 118 | |
| YAR | 0402:0302 | 2859 | — | — | |
| MZ070782 | 0102:0501 | 3000 | — | — | |
| PE117 | 0404:0302 | 6238 | — | — | |
| DEU | 0401:0301 | 2182 | — | — | |
| | M.C. T cell line | | | | |
| PBMC | 1501/1302:0602/0604 | 864 | 32 | | 28 |
| HHKB | 1301:0603 | 749 | — | | — |
| KAS011 | 1601:0502 | 961 | — | | — |
| CMW | 1301:0603 | 792 | — | | — |
| E4181324 | 1502:0601 | 896 | — | | 34 |
| WT47 | 1302:0604 | 526 | — | | — |
| WT8 | 1501:0602 | 1079 | 11 | | 41 |
| HO31 | 1302:0604 | 1300 | — | | — |
| EA | 1501:0602 | 3298 | 13 | | 12 |

### EXAMPLE 3

### Identification of the Autoreactive Peptide p556-p575

Analogous to the procedure described in example 1.4 a screening was carried out for further autoreactive peptides from the human GAD 65 kd. In this case it was found that the T cell line 40/2 was reacted with an individual peptide pool. When examining individual peptides of this peptide pool it was found that the T cell line 40/2 exclusively reacted with the peptide p556-575.

In order to determine the isotype of MHC molecules which present the autoreactive peptide p556-575, autologous PBLs were preincubated with monoclonal antibodies which recognize HLA-DR, HLA-DQ and HLA class I molecules. Peptide p556-575 was then added. The T cells were added after an intermediate incubated of 3 hours and a proliferation test was carried out. In this process it was found that a significant inhibition of proliferation only occurs in the presence of the monoclonal antibody which recognizes HLA-DR. Since the patient which has been derived from the T cell line 40/2 expressed the allel DRB1*0401 this is therefore identified as a restriction element.

### EXAMPLE 4

### Identification of T Cell Receptors (TCR)

Total RNA was isolated from T cells in order to identify and sequence GAD-specific TCR. For this the cells in suspension were washed with PBS and the cell pellet was resuspended with 0.2 ml RNAzol-B per 1×10⁶ cells.

After mechanically resuspending the lysates several times and optionally adding yeast tRNA as a carrier matrix, the RNA was extracted by addition of 0.2 ml chloroform per 2 ml homogenate, subsequently mixing for 15 sec. and storing for 5 minutes on ice.

After a centrifugation step of 12,000 g for 15 min. the aqueous phase was removed and transferred into a new reaction vessel. The first precipitation of the RNA was achieved by addition of an identical volume of isopropanol and subsequent storage for at least 15 min. at 4° C. After centrifugation for 15 min. at 12,000 g and 4° C. the RNA was obtained as a pellet at the bottom of the vessel.

After discarding the supernatant the RNA pellet was purified of salts by briefly mixing in 75% ethanol. After centrifugation (7,500 g, 4° C., 8 min) the pellet was dissolved in 100 μl water that had been treated with diethyl pyrocarbonate (DEPC) and again precipitated with 250 μl ethanol and 10 μl 2 M NaCl for at least 1 h at −20° C. The centrifugation and washing steps after the second precipitation were carried out as described for the first precipitation. After drying the pellet in air the RNA was resuspended in H₂O-DEPC.

cDNA was synthesized from the RNA by reverse transcription. For this ca. 3 μg total RNA was incubated for 10 min at 55° C. with 30 ng p-CαST (a specific primer for the TCR α chain having the sequence 5′-CAC TGA AGA TCC ATC ATC TG-3′) and 30 ng p-CβST (a specific primer for the β chain having the sequence 5′-TAG AGG ATG GTG GCA GAC AG-3′) in a reaction volume of 10 μl. Subsequently 38 μl RAV-2-RT buffer (100 mM Tris-HCl pH

8.3; 140 mM KCl, 10 mM MgCl₂; 2 mM dithiothreitol, 0.1 mM of each dNTP), 1 μl (0.75 U) rRNasin and 1 μl (18 U) reverse transcriptase were added by pipette. The reverse transcription was carried out for 90 min. at 42° C. followed by a denaturation step at 68° C. for 5 min. It was stored at −80° C. until use.

Subsequently a polymerase chain reaction (PCR) was carried out. Whether the corresponding V family was expressed or not was indicated by the occurrence of an amplificate using 5′-family specific primers for the variable domains of the α and β chains. The 3′ primers were located in the constant domain and were the same in all α and β preparations. A control amplificate which is located in the constant domain and does not overlap the specific amplification product indicates whether the PCR reaction has worked in this preparation and could be used for the semi-quantitative determination of V-family specific expression.

The primers were also used in a biotinylated form in order to enable a subsequent purification of the PCR products by coupling to a magnetic particulate solid phase (streptavidin-coated beads).

The PCR was carried out using a thermostable DNA polymerase with the following reaction scheme:

| | | | |
|---|---|---|---|
| 94° C. | 4 | min. | predenaturation |
| 94° C. | 30 | sec. | DNA denaturation |
| 56° C. | 30 | sec. | annealing |
| 72° C. | 1 | min. | extension |
| 72° C. | 5 | min. | filling up all single strands in the |
| | | | reaction solution (only at the end). |

The number of reaction cycles in the PCR was usually 35.

The PCR fragments obtained in this manner were sequenced.

The 4 independently isolated GAD-specific T cell clones of patient 24: 24/31#1/1, 24/31#1/4, 24/31#9, 24/31#PF7 all expressed the same TCR. This is composed of: Vβ8 (AV8S1A1) and Vβ5 (BV5S1A1T). The J gene segments and the CDR3 regions used are also identical.

The T cell clone 40/2#20 of patient 40 expresses 2 α chains, i.e. Vα2 (AV2S1A2) and Vα21 (ADV21S1A1) and a Vβ chain Vβ2 (BV2S1A4T).

The sequence data of the CDR3 regions from the TCR a and TCR3 β chains are shown in FIGS. 5 and 6.

The complete sequences of the TCR can be determined without difficulty with the aid of known sequences from the GENBank/EMBL data bank. The respective accession numbers are as follows:

| | | |
|---|---|---|
| Vα8 | (AV8S1A1) | X04954/M13734 |
| Vα2 | (AV2S1A2) | M17652 |
| Vα21 | (ADV21S1A1) | M15565 |
| Vβ5 | (BV5S1A1T) | X04954 |
| Vβ2 | (BV2S1A4T) | M11954 |

1. Peptide or peptide derivative comprising:
(a) the amino acid sequence (I)
D-V-N-Y-A-F-L-H-A-T-D-L-L-P-A-C-D-G-E-R,
(b) the amino acid sequence (II)
S-N-M-Y-A-M-M-I-A-R-F-K-M-F-P-E-V-K-E-K,
(c) the amino acid sequence (III)
N-W-E-L-A-D-Q-P-Q-N-L-E-E-I-L-M-H-C-Q-T,
(d) the amino acid sequence (IV)
T-L-K-Y-A-I-K-T-G-H-P-R-Y-F-N-Q-L-S-T-G,
(e) the amino acid sequence (V)
P-R-Y-F-N-Q-L-S-T-G-L-D-M-V-G-L-A-A-D-W,
(f) the amino acid sequence (VI)
T-Y-E-I-A-P-V-F-V-L-L-E-Y-V-T-L-K-K-M-R,
(g) Amino acid sequence (VII)
F-F-R-M-V-I-S-N-P-A-A-T-H-Q-D-I-D-F-L-I,
(h) partial regions of the amino acid sequence shown in (a), (b), (c), (d), (e), (f) or/and (g) with a length of at least 6 amino acids or/and
(i) amino acid sequences which have an essentially equivalent specificity or/and affinity of binding to MHC molecules as the amino acid sequences shown in (a), (b), (c), (d), (e), (f), (g) or/and (h).

2. Peptide or peptide derivative as claimed in claim 1,
wherein
it has at least a length of eight amino acids.

3. Peptide or peptide derivative as claimed in claim 1 or 2,
wherein
it has at least a length of 10 amino acids.

4. Peptide or peptide derivative as claimed in one of the claims 1 to 3,
wherein
it has a length of up to 25 amino acids.

5. Peptide or peptide derivative as claimed in one of the claims 1 to 4,
wherein
it carries a marker group.

6. Peptide mimetic,
wherein
it has an essentially equivalent specificity or/and affinity of binding to MHC molecules as a peptide or peptide derivative as claimed in one of the claims 1 to 5.

7. Complex which at least comprises a peptide or peptide derivative as claimed in one of the claims 1 to 5 or a peptide mimetic asa peptide or peptide derivative as claimed in one of the claims 1 to 5.

7. Complex which at least comprises a peptide or peptide derivative as claimed in one of the claims 1 to 5 or a peptide mimetic as claimed in claim 6 which is bound to a MHC molecule or a peptide-binding derivative of a MHC molecule.

8. Complex as claimed in claim 7,
wherein
it comprises a MHC class II molecule or a peptide-binding derivative thereof.

9. Complex as claimed in claim 8,
wherein
it has a MHC class II molecules of types DR1, DR2, DR4 or DQ6.

10. Complex as claimed in claim 9,
wherein
the MHC class II molecule has the subtype DR B1*101, DR B1*1501, DR B1*1502, DR B1*1601, DR B5*0101, DR B1*0401 or DQ B1*0602.

11. Complex as claimed in one of the claims 7 to 10,
wherein
it comprises a recombinant MHC molecule or a peptide-binding derivative thereof.

12. Complex as claimed in claim 11,
wherein
it comprises a soluble peptide-binding derivative of a MHC molecule.

13. Complex as claimed in one of the claims 7 to 12,
wherein
it carries a marker group.

14. Complex as claimed in one of the claims 7 to 13,
wherein
it at least contains 2 MHC molecules or MHC molecule derivatives which are associated by covalent or non-covalent interactions.

15. Complex as claimed in claim 24,
wherein
it contains peptide MHC molecule complexes that are cross-linked by chemical coupling reagents.

16. Complex as claimed in claim 14,
wherein
it contains MHC molecules or MHC molecule derivatives that are cross-linked with several MHC-binding regions via an oligomerized peptide component.

17. Complex as claimed in claim 14,
wherein
it contains peptide-MHC molecule complexes that are cross-linked by antibodies.

18. Pharmaceutical composition,
wherein
it contains a peptide or peptide derivative as claimed in one of the claims 1 to 5, a peptide mimetic as claimed in claim 6 or/and a complex as claimed in one of the claims 7 to 17 as the active component if desired in combination with common pharmaceutical additives.

19. Composition as claimed in claim 18,
wherein
it in addition comprises an accessory-stimulating component.

20. Composition as claimed in claim 19,
wherein
the accessory-stimulating component is selected from cytokines or/and the surface antigen B7.

21. Use of a pharmaceutical composition as claimed in one of the claims 18 to 20 for the production of an agent for the diagnosis of diseases or a predisposition for diseases which influence the immune system or for the diagnosis of tumour diseases or a predisposition of tumour diseases.

22. Use as claimed in claim 21 for the production of an agent for the diagnosis of autoimmune diseases or a predisposition of autoimmune diseases.

23. Use as claimed in claim 21 or 22 for the production of an agent for the diagnosis of diabetes or a predisposition of diabetes.

24. Method for the determination of a specific T cell subpopulation,
wherein
a sample containing T cells is contacted with a peptide or peptide derivative as claimed in one of the claims 1 to 5, a peptide mimetic as claimed in claim 6 or/and a complex as claimed in one of the claims 7 to 17 and the reaction of T cells with the peptide or complex is determined in the sample.

25. Method as claimed in claim 24,
wherein
the reaction of the T cells with a fluorescent-labelled peptide or complex is determined by FACS analysis.

26. Method as claimed in claim 24 or 25,
wherein
preactivated T cells are selected before or/and after contacting the T cells with the peptide or the complex.

27. Use of a pharmaceutical composition as claimed in one of the claims 18 to 20 for the production of an agent for therapy or prevention of diseases which influence the immune system.

28. Use as claimed in claim 27 for the production of an agent for the therapy or prevention of autoimmune diseases.

29. Use as claimed in claim 27 or 28 for the production of an agent for the therapy or prevention of diabetes.

30. Use of a peptide or peptide derivative as claimed in one of the claims 1 to 5, a peptide mimetic as claims in claim 6 or a complex as claimed in one of the claims 7 to 17 for the production of an antigen in particular an immunogen or tolerogen.

31. Method for the isolation of a specific T cell subpopulation,
wherein
a sample containing T cells is contacted with a peptide or peptide derivative as claimed in one of the claims 2 to 5, a peptide mimetic as claimed in claim 6 or a complex as claimed in one of the claims 7 to 17, the T cells that react with the peptide or complex are identified and separated from other T cells if desired.

32. Method as claimed in claim 31,
wherein
preactivated T cells are selected before or/and after contacting the T cells with the peptide or the complex.

33. Use of T cells isolated according to the method as claimed in claim 31 or partial structures thereof for the production of an antigen.

34. Use as claimed in claim 33,
wherein
the T cells or partial structures thereof are re-injected into the patients from whom they are originally derived.

35. Use as claimed in claim 34,
wherein
inactivated T cells are reinjected.

36. Use as claimed in claim 35,
wherein
T cells capable of division are reinjected.

37. Antibody against a peptide or peptide derivative as claimed in one of the claims 1 to 5, a peptide mimetic as claimed in claim 6 or a complex as claimed in one of the claims 7 to 17, obtainable by immunization with a peptide, peptide derivative, peptide mimetic or complex and isolating an antibody produced by the immunization.

38. Anti-idiotypic antibody against an antibody as claimed in claim 37, obtainable by immunizing the antibody against the peptide, peptide derivative or peptide mimetic or the complex and isolating an anti-idiotypic antibody produced by the immunization.

39. T cell which reacts with a peptide or peptide derivative as claimed in one of the claims 1 to 3, a peptide mimetic as claimed in claim 6 or a comples as claimed in one of the claims 7 to 17.

40. Use of peptides of glutamic acid decarboxylase (GAD) peptide derivatives derived therefrom or peptide mimetics for the production of a pharmaceutical agent which leads to the formation of an immune tolerance when administered to patients with diabetes.

41. Use as claimed in claim 40,
wherein
the peptides, peptide derivatives or peptide mimetics are administered at a dose of 3 to 30 mg per kg body weight.

42. Use as claimed in claim 40 or 41,
wherein
at least a second vaccination is carried out after administration of the peptides, peptide derivatives or peptide mimetics.

43. Use as claimed in one of the claims 40 to 42,
wherein
in the second or optionally following vaccinations peptides, peptide derivatives or peptide mimetic complete GAD or/and a part thereof containing the sequence of the peptides which have already been used in the first vaccination are used.

44. Use as claimed in claim 43,
wherein
the vaccinations are carried out each at intervals of 7 to 14 days.

45. Use as claimed in one of the claims 40 to 44,
wherein
a mixture of various peptides, peptide derivatives or peptide mimetic is used.

46. T cell,
wherein
it contains a T cell receptor which binds to a peptide or peptide derivative as claimed in one of the claims 1 to 5, to a peptide mimetic as claimed in claim 6 or to a complex as claimed in one of the claims 7 to 17.

47. T cell as claimed in claim 46,
wherein
it has a T cell receptor which comprises a TCRα chain containing a CDR3 region shown in FIG. 5 or one that is at least 70% homologous thereto or/and a TCRβ chain containing a CDR3 region shown in FIG. 6 or one that is at least 70% homologous thereto.

48. Polypeptide with T cell receptor activity,
wherein
it binds to a peptide or peptide derivative as claimed in one of the claims 1 to 5, to a peptide mimetic as claimed in claim 6 or to a complex as claimed in one of the claims 7 to 17.

49. Polypeptide as claimed in claim 48,
wherein
it comprises a TCRα chain containing a CDR3 region shown in FIG. 5 or an amino acid sequence that is at least 70% homologous thereto.

50. Polypeptide as claimed in claim 48 or 49,
wherein
it comprises a TCRβ chain containing a CDR3 region shown in FIG. 6 or an amino acid sequence that is at least 70% homologous thereto.

51. Nucleic acid,
wherein
it codes for a polypeptide as claimed in one of the claims 48 to 50.
